(19) United States
(12) Patent Application Publication
(10) Pub. No.: US 2014/0243416 A1
(43) Pub. Date: Aug. 28, 2014

(54) Topical Antiandrogen Therapy for the Treatment of Becker's Nevus

(71) Applicants: Arash Taheri, Isfahan (IR); Parisa Mansoori, Isfahan (IR); Laura F. Sandoval, Winston Salem, NC (US); Steven R. Feldman, Winston Salem, NC (US)

(72) Inventors: Arash Taheri, Isfahan (IR); Parisa Mansoori, Isfahan (IR); Laura F. Sandoval, Winston Salem, NC (US); Steven R. Feldman, Winston Salem, NC (US)

(21) Appl. No.: 13/779,760

(22) Filed: Feb. 28, 2013

(51) Int. Cl. A61K 31/167; A61K 9/00

(52) U.S. Cl. CPC A61K 31/167; A61K 9/0014
USPC 514/629

(57) ABSTRACT

This invention relates to the treatment of Beckers nevus with antiandrogenic topical agents, including 5-alpha reductase inhibitors, non-steroidal antiandrongens and steroidal antiandrogens.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

The present invention relates to the topical application of antiandrogen for the treatment of Becker's nevus.

Becker's nevus is a benign lesion characterized by a hyperpigmented patch with a sharp and irregular border. Frequently this nevus has an associated hypertrichosis with darker and thicker hairs compared to uninvolved skin. Becker's nevus is considered to be a congenital anomaly.

Becker's nevus is thought to be an androgen-dependent lesion. Though onset can be as early as immediately after birth, this nevus generally appears around puberty, becomes more prominent after puberty, and tends to be more conspicuous in male patients. Acne vulgaris may be more prevalent or more severe on the Becker's nevus, it has been reported in association with an accessory scrotum, and in women the most common anomaly is ipsilateral mammary hypoplasia, all in support of this lesion being androgen-dependent (Szylit 1986; Danarti 2004). Furthermore, tinea versicolor shows a particular affinity to Becker's nevus, which may be due to an increased sebum production. (Wright 1979) Studies show an increased number of androgen receptors and androgen receptor messenger RNA in Becker's nevus compared to unaffected skin. (Person 1984; Nirde 1999; Formigon 1992).

Becker's nevus does not cause any symptom or morbidity in most of the patients, and malignant transformation has not been reported; however, this hairy hyperpimented lesion is sometimes cosmetically unacceptable and may have psychosocial implications.

Becker's nevus tend to be difficult to treat. Treatment of the pigmented portion of the lesion has shown improvement with various laser modalities such as Q-switch, Er-YAG, and 1,550 nm fractional erbium-doped fiber laser. (Polder 2011)

Seeking to target the androgenic pathogenesis of Becker's nevus, a topical antiandrogen, specifically topical flutamide was choosen. Flutamide is a non-steroidal antiandrogen that competes with androgens for binding to androgen receptors. Topical flutamide is absorbed into the skin and systemic absorption is measurable. (Katchen 1976) It has shown some effects in treatment of acne vulgaris and androgenetic alopecia. (Gwiezdzinski 1997; Sintov 2000). Similar non-steroidal antiandrogens include nilutamide, bicalutamide, and enzalutamide, which have the potential to be formulated into topical formulations to target the androgenic pathogenesis of Becker's nevus. Newer non-steroidal antiandrogens include CYP17 inhibitors such as abiraterone, with galeterone, ARN-509, and orteronel in development, suppress androgen synthesis (Schweizer 2012). Steroidal antiandrogen agents ciproterone acetate, megastrol acetate, and spironolactone and 5-alpha reductase inhibitors finasteride, dutasteride, and alfatradiol all possess antiandrogen activity and could be used topically to treat Becker's nevus.

### REFERENCES

Danarti R, König A, Salhi A, Bittar M, Happle R. Becker's nevus syndrome revisited. J Am Acad Dermatol. 2004 Dec;51(6):965-9.
Formigon et al. Becker's nevus and ipsilateral breast hypoplasia: androgen-receptor study in two patients. Arch Dermatol 1992;128:992-3.
Gwiezdzinski et al. 2.5% solution of flutamide (a nonsteroidal antiandrogen) in the topical treatment of acne vulgaris. A double blind randomized study. Journal of Dermatological Treatment 1997, Vol. 8, No. 2 , Pages 75-78.
Katchen et al. Percutaneous penetration and metabolism of topical (14C) flutamide in men. J Invest Dermatol. 1976 June;66(6):379-82.
Nirde et al. The association of Becker nevus with hypersensitivity to androgens. Arch Dermatol 1999;135:212-4.
Person et al. Becker's nevus: an androgen-mediated hyperplasia with increased androgen receptors. J Am Acad Dermatol 1984;10:235-8
Polder et al. Laser eradication of pigmented lesions: a review. Dermatol Surg. 2011 May; 37(5):572-95.)
Schweizer et al. Abiraterone and other novel androgendirected strategies for the treatment of prostate cancer: a new era of hormonal therapies is born Ther Adv Urol. 2012 August; 4(4) 167-178.
Sintov et al. New topical antiandrogenic formulations can stimulate hair growth in human bald scalp grafted onto mice. Int J Pharm. 2000 Jan. 20; 194(1):125-34.
Szylit J A, Grossman M E, Luyando Y, Olarte M R, Nagler H. Becker's nevus and an accessory scrotum: a unique occurrence. J Am Acad Dermatol 1986; 14:905-7.
Wright R C. Another association with Becker's nevus. Arch Dermatol 1979;115:1035.

### BRIEF SUMMARY OF THE INVENTION

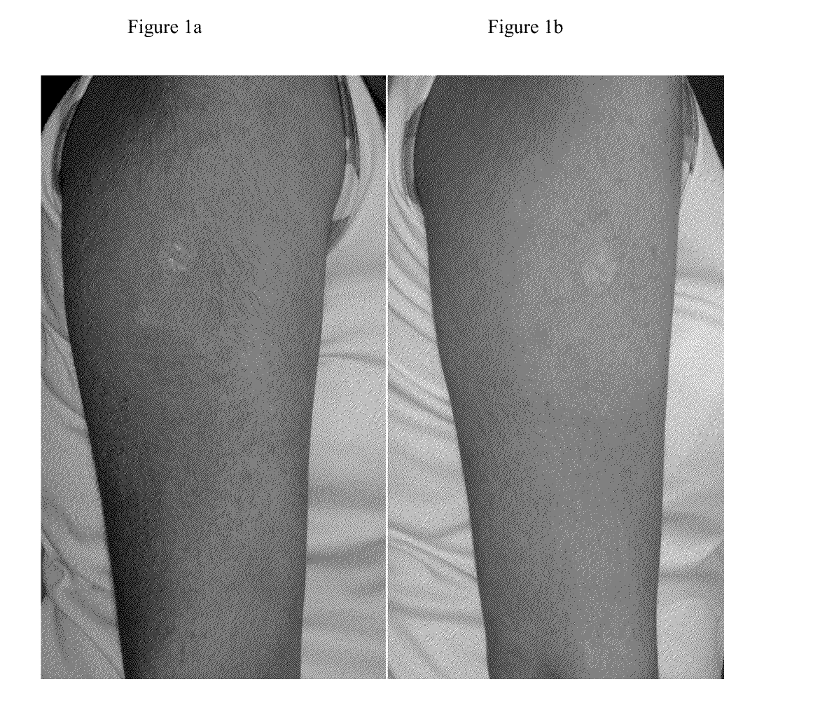

The present invention represents a method of treating Becker's nevus with topical antiandrogens, including 5-alpha reductase inhibitors, non-steroidal antiandrongens and steroidal antiandrogens. Specifically, in the example hereinafter presented, it has been demonstrated that the topical application of flutamide results in the reduction in the hyperpigmentation of the lesion. (FIGS. 1a, 1b)

The method of treatment according to this invention is the application of 0.5%-5% by weight of topical antiandrogen, in the form of a cream, a lotion, a gel, an ointment, a solution, or a serum.

### BRIEF DESCRIPTION OF THE SEVERAL VIEWS OF THE DRAWING

### Brief descriptions of the Figures

FIG. 1a (left) depicts a woman with a Becker's nevus before the antiandrogen treatment as described in example 1.

FIG. 1b (right) depicts a woman after treatment with a topical antiandrogen, specifically topical flutamide. A reduction in hyperpigmentation of the lesion occurred after 8 weeks of treatment as described in example 1.

### DETAILED DESCRIPTION OF THE INVENTION

The present invention provides a method for treating Becker's nevus by applying topical antiandrogens. By topically applying substances which are inhibitory in terms of production and/or effect of androgens including 5-alpha reductase inhibitors, non-steroidal antiandrongenic agents, including but not limited to topical flutamide, and steroidal antiandrogenic agents, the method results in a reduction in the hyperpigmentation of the lesion.

The present invention is directed to a method of reducing the hyperpigmentation of the Becker's nevus lesion with the administration of 0.5%-5% by weight of active antiandrogen.

The treatment according to this invention can be applied topically daily or twice daily. The treatment can be continuous for 30 days, 60 days, 12 weeks, 6 months, or as long as the patients requires treatment to achieve the desired reduction in hyperpigmentation.

The dosage form can be formulated into a pharmaceutically acceptable form according to conventional techniques. This form includes but is not limited to a cream, a lotion, a gel, an ointment, a solution, or a serum. The active topical antiandrogen can be formulated by combining it with pharmaceutically acceptable inactive agents, including but not limited to emollients, water soluble vehicles, moisturizers, preservatives, and the likes. Inactive agent refers to a substance that by itself has no therapeutic value. Compositions of the present invention may also contain additional inactive agents such absorption enhancers which may modify the penetration rate of the active agent into the skin and provide an increased and longer lasting availability of the active agent on the skin.

The amount of antiandrogen agent in the dosage form can vary. For example, the dosage form can contain between 0.5% to 5% by weight of active agent.

The compositions according to the present invention may contain one or more active agents selected from 5-alpha reductase inhibitors, non-steroidal and steroidal antiandrogens.

Examples of 5-alpha reductase inhibitors which may be included in the composition in accordance with the present invention include N-(1,1-dimethylethyl)-3-oxo-(5α, 17β)-4-azaandrost-1-ene-17-carboxamide (finasteride), (5α, 17β)-N-{2,5 bis(trifluoromethyl) phenyl}-3-oxo-4-azaandrost-1-ene-17-carboxamide (dutasteride), and estra-1,3,5(10)-triene-3,17α-diol (alfatradiol).

Examples of non-steroidal antiandrogens which may be included in the composition in accordance with the present invention include methyl-N-[4-nitro-3-(trifluoromethyl)phenyl]-propanamide (flutamide), 1-[4-(4-{[(2R,4S)-2-(2,4-Dichlorophenyl)-2-(1H-imidazol-1-ylmethyl)-1,3-dioxolan-4-yl]methoxy}phenyl) piperazin-1-yl]ethan-1-one (ketoconazole), 5,5-dimethyl-3-[4-nitro-3-(trifluoromethyl) phenyl]imidazolidine-2,4-dione (nilutamide), N-[4-cyano-3-(trifluoromethyl) phenyl]-3-[(4-fluorophenyl)sulfonyl]-2-hydroxy-2-methylpropanamide (bicalutamide), (3β)-17-(pyridin-3-yl)androsta-5,16-dien-3-ol(abiraterone), 6-(7-Hydroxy-6,7-dihydro-5H-pyrrolo[1,2-c]imidazol-7-yl)-N-methylnaphthalene-2-carboxamide (orteronel,TAK-700), 17-(1H-benzimidazol-1-ypandrosta-5,16-dien-3β-ol (galeterone, TOK-001), 4-(3-(4-cyano-3-(trifluoromethyl) phenyl)-5,5-dimethyl-4-oxo-2-thioxoimidazolidin-1-yl)-2-fluoro-N-methylbenzamide (enzalutamide), 4-(7-(6-cyano-5-(trifluoromethyl)pyridin-3-yl)-8-oxo-6-thioxo-5,7-diazaspiro[3.4]octan-5-yl)-2-fluoro-N-methylbenzamide (ARN-509).

Examples of steroidal antiandrogens which may be included in the composition in accordance with the present invention include 6-chloro-17-hydroxy-1a,2a-methylenepregna-4,6-diene-3,20-dione (ciproterone acetate), 17-(acetyloxy)-6-methyl-pregna-4,6-diene-3,20-dione (megastrol acetate), and 7α-acetylthio-3-oxo-17α-pregn-4-ene-21,17-carbolactone or 17-Hydroxy-7a-mercapto-3-oxo-17a-pregn-4-ene-21-carboxylic acid g-lactone acetate (spironolactone).

The present invention is illustrated by the following example. It should, however, be noted that such example is given by way of illustration and not of limitation.

### Example 1

A topical 4% solution of flutamide was applied twice daily to a Becker's nevus. After eight weeks of therapy, the hyperpigmentation was significantly reduced. After 30 weeks of topical flutamide use, there was no further appreciable improvement in the lesion compared to the eighth week of treatment. No significant change to the hypertrichosis in the area was observed. There was no pigmentary change in the normal skin surrounding the Becker's nevus during treatment. No cutaneous or systemic adverse effects were reported and the patient denied any menstrual irregularities. (FIG. 1)

Invention claim:

1. A method of treating Becker's nevus consisting of applying a therapeutically effective dose of topical antiandrogen from one or more active agents selected from the groups consisting of 5-alpha reductase inhibitors, non-steroidal antiandrogenenic agents, and steroidal antiandrogenic agents.

2. The method of claim 1, wherein there is a decrease in the hyperpigmentation of the Becker's nevus lesion.

3. The method of claim 1, wherein the preparation comprises from about 0.5% to about 5% by weight of active antiandrogen agent.

4. The method of claim 3, wherein the topical antiandrogen preparation is applied once or twice daily.

5. The method of claim 3, wherein the preparation is present as a lotion, a cream, a gel, an ointment, a solution, or a serum.
